(19) United States
(12) Patent Application Publication
(10) Pub. No.: US 2023/0293755 A1
(43) Pub. Date: Sep. 21, 2023

(54) INACTIVATING APPARATUS AND LIGHT IRRADIATION DEVICE

(71) Applicant: Ushio Denki Kabushiki Kaisha, Tokyo (JP)

(72) Inventors: Akira ISHIKURA, Tokyo (JP); Koji YAMADA, Tokyo (JP)

(73) Assignee: Ushio Denki Kabushiki Kaisha, Tokyo (JP)

(21) Appl. No.: 18/175,382

(22) Filed: Feb. 27, 2023

(30) Foreign Application Priority Data
Mar. 16, 2022 (JP) 2022-041165

(51) Int. Cl. A61L 2/24; A61L 2/10; G01J 5/00; G01S 15/08

(52) U.S. Cl. CPC A61L 2/24; A61L 2/10; G01J 5/0025; G01S 15/08; A61L 2202/11; A61L 2202/16; A61L 2202/14

(57) ABSTRACT

Providing an inactivating apparatus for inactivating pathogens using ultraviolet light which has improved convenience of use. The inactivating apparatus including: a light irradiation device having a light source that emits ultraviolet light for inactivating pathogens; a fixing base to/from which a main body of the light irradiation device is attachable/detachable; and a controller that switches control of lighting state of the light irradiation device between when the main body is attached to the fixing base and when the main body is detached from the fixing base.

### CROSS-REFERENCE TO RELATED APPLICATIONS

This application claims priority of Japan Patent Application No. 2022-041165, which was filed on Mar. 16, 2022, and which is incorporated herein in its entirety by reference.

### BACKGROUND OF THE INVENTION

### Field of the Invention

The present invention relates to an inactivating apparatus and a light irradiation device for inactivating pathogens.

### Description of the Related Art

An inactivating apparatus for inactivating pathogens such as bacteria and viruses using ultraviolet light is conventionally known. Such inactivating apparatuses include not only an inactivating apparatus fixed to a ceiling to radiate ultraviolet light to a space but also a portable inactivating apparatus carried and used by a user (see Patent Document 1).

### PRIOR ART DOCUMENT

### Patent Document

Patent Document 1: Japanese Translation of PCT International Application Publication No. JP-A-2021-514287

### SUMMARY OF THE INVENTION

Nowadays, due to the COVID-19 epidemic, there is a growing need for inactivation of pathogens such as viruses. It is therefore an object of the present invention to provide an inactivating apparatus for inactivating pathogens using ultraviolet light which has improved convenience of use.

The present invention is directed to an inactivating apparatus including:
a light irradiation device having a light source that emits ultraviolet light for inactivating pathogens;
a fixing base to/from which a main body of the light irradiation device is attachable/detachable; and
a controller that switches control of lighting state of the light irradiation device between when the main body is attached to the fixing base and when the main body is detached from the fixing base.

The inactivating apparatus is used in two ways: one is a method in which the light irradiation device is used by being attached to the fixing base; and the other is a method in which the light irradiation device is used by being detached from the fixing base and carried by a user. Therefore, the inactivating apparatus has high convenience of use. Further, the inactivating apparatus can switch a lighting state between when the light irradiation device is used by being attached to the fixing base and when the light irradiation device is carried and used by a user. Therefore, a suitable lighting state can be selected depending on how the light irradiation device is used, which makes it possible to use the inactivating apparatus more safely and effectively.

The term “pathogens” as used herein refers to microorganisms such as bacteria, fungi, and viruses. Pathogens are present on the surfaces of human bodies (e.g., skins and hairs) and the surfaces of objects that humans frequently touch (e.g., furniture, office equipment, floors, or walls) or are suspended in spaces where humans are present. Particularly, viruses are often contained in airborne droplets released from the mouths or noses of humans or animals and spread through expired air or saliva or by coughing or sneezing or in aerosols. Therefore, viruses are particularly suspended widely in spaces and attached to the surfaces of objects such as furniture, floors, and walls and the surfaces of human bodies.

In the case of protozoa, the term “inactivate” as used herein refers to either killing them by, for example, destroying DNA or enzymes (proteins) in cells or cell membranes or depriving cells of their proliferative function. In the case of viruses, the term “inactivate” as used herein refers to depriving them of their infectivity in cells by destroying DNA or RNA.

Ultraviolet light inactivates pathogens. Particularly, ultraviolet light having an emission wavelength of 190 nm or more and less than 240 nm is safe for human bodies and therefore can be radiated to human bodies. Therefore, the inactivating apparatus should use a light source that radiates ultraviolet light having an emission wavelength of 190 nm or more and less than 240 nm. However, as in the case of, for example, a fourth embodiment that will be described later, a light source that does not radiate ultraviolet light having an emission wavelength of 190 nm or more and less than 240 nm may be used in some embodiments of the inactivating apparatus.

The light irradiation device may further have a lighting switch for turning on the light source, and

the controller activates a first control mode irrespective of a state of the lighting switch when the light irradiation device is attached to the fixing base, and activates a second control mode for a higher illuminance than the first control mode when the light irradiation device is detached from the fixing base and a user of the inactivating apparatus turns on the lighting switch, the controller.

In the first control mode, the controller may control irradiation intensity and irradiation time of the ultraviolet light so that a specified value is not exceeded, and in the second control mode, the controller may control irradiation intensity of the light source so that the specified value is exceeded.

The light irradiation device may have a detector for detecting detaching of the light irradiation device from the fixing base. The detection result of the detector is used to switch the control mode of the controller. As the detector, for example, a push-button type switch may be provided.

The light irradiation device may further have a human sensor, and

when the human sensor detects a human in a second control mode, the light source is not turned on. This makes it possible to further increase safety.

The light irradiation device may further have a distance sensor, and

the controller turns on the light source when the distance sensor does not detect an object nearer than a first specified value in a first control mode, and turns on the light source when the distance sensor detects an object nearer than a second specified value in a second control mode. Although described later in detail, in this case, the first control mode and the second control mode are opposite in the lighting control of the light irradiation device 1 when the distance sensor detects/does not detect an object.

A light distribution angle of emitted light when the light irradiation device is attached to the fixing base may be larger than that when the light irradiation device is detached from the fixing base.

The fixing base may have a light diffuser, and

when the light irradiation device is attached to the fixing base, the light distribution angle may be increased by passage of emitted light from the light irradiation device through the light diffuser. In this case, when the light irradiation device is used by being attached to the fixing base, the ultraviolet light is diffused, and when the light irradiation device is carried, the light irradiation device can be used without diffusing the ultraviolet light.

Irradiation intensity of the ultraviolet light when the light irradiation device is attached to the fixing base may be lower than that when the light irradiation device is detached from the fixing base.

The fixing base may have a neutral density filter, and

when the light irradiation device is attached to the fixing base, irradiation intensity of emitted light from the light irradiation device is reduced by passing through the neutral density filter. In this case, when the light irradiation device is used by being attached to the fixing base, the ultraviolet light can be reduced, and when the light irradiation device is carried, the light irradiation device can be used without reducing the ultraviolet light.

The light irradiation device may further have a handle separable from the main body of the light irradiation device, and

the handle may have a storage battery for turning on the light source.

The fixing base may be disposed on a table, a floor, a wall, a column, or a ceiling or may be disposed in equipment for holding the light irradiation device. Alternatively, the fixing base may be part of a table, a floor, a wall, a column, or a ceiling or may be part of equipment for holding the main body of light irradiation device.

When the light irradiation device is attached to the fixing base, emitted light may be radiated to an environment isolated from a space where a human is present. The environment isolated from a space where a human is present is, for example, a space where a human is absent. Such spaces include the interior of housing of an air purifier or an air conditioner.

The present invention is also directed to a light irradiation device having a light source that emits ultraviolet light for inactivating pathogens, the light irradiation device including:
a light source that emits ultraviolet light for inactivating pathogens; and
a controller that switches control of lighting state of the light irradiation device between when the light irradiation device is attached to the fixing base and when the light irradiation device is detached from the fixing base, and being configured to be attachable to/detachable from the fixing base.

The present invention is also directed to an inactivating apparatus including:
a light irradiation device having a light source that emits ultraviolet light for inactivating pathogens; and
a fixing base to/from which the light irradiation device is attachable/detachable and which has at least one of a light diffuser and a neutral density filter,
wherein when the light irradiation device is attached to the fixing base, emitted light from the light irradiation device passes through at least one of the light diffuser and the neutral density filter. In this case, when the light irradiation device is used by being attached to the fixing base, the ultraviolet light can be diffused or reduced, and when the light irradiation device is carried, the light irradiation device can be used without diffusing or reducing the ultraviolet light.

It is possible to provide an inactivating apparatus having improved convenience of use. Providing such an inactivating apparatus responds to Goal 3 “Ensure healthy lives and promote well-being for all at all ages” of Sustainable Development Goals (SDGs) led by United Nations and greatly contributes to Target 3.3 “By 2030, end the epidemics of AIDS, tuberculosis, malaria and neglected tropical diseases and combat hepatitis, water-borne diseases and other communicable diseases”.

### BRIEF DESCRIPTION OF THE DRAWINGS

FIG. 1 is an external view when an inactivating apparatus according to a first embodiment is used by fixing a light irradiation device;

FIG. 2 is an external view when the inactivating apparatus according to the first embodiment is used by carrying the light irradiation device;

FIG. 3A is a block diagram when the inactivating apparatus according to the first embodiment is used by fixing the light irradiation device;

FIG. 3B is a block diagram when the inactivating apparatus according to the first embodiment is used by carrying the light irradiation device;

FIG. 4A is a flow chart of control of the inactivating apparatus according to the first embodiment;

FIG. 4B is a flow chart of control of the inactivating apparatus according to the first embodiment;

FIG. 5 is an external view when an inactivating apparatus according to a second embodiment is used by fixing a light irradiation device;

FIG. 6 is an external view when the inactivating apparatus according to the second embodiment is used by carrying the light irradiation device;

FIG. 7 is an external view when an inactivating apparatus according to a third embodiment is used by fixing a light irradiation device;

FIG. 8A is an external view of an air purifier including an inactivating apparatus according to a fourth embodiment; and

FIG. 8B is a conceptual diagram of the air purifier including the inactivating apparatus according to the fourth embodiment.

### DETAILED DESCRIPTION OF THE PREFERRED EMBODIMENTS

Embodiments of an inactivating apparatus will be described with reference to the drawings. It should be noted that the drawings disclosed herein merely show schematic illustrations. Namely, the dimensional ratios on the drawings do not necessarily reflect the actual dimensional ratios, and the dimensional ratios are not necessarily the same between the drawings.

Hereinafter, some of the drawings will be described with reference to an XYZ coordinate system. In the following description, in a case where positive and negative directions are distinguished from each other when a direction is expressed, the direction is described using a positive or negative sign, such as “+X direction” or “−X direction”. In the case of expressing a direction without distinguishing between positive and negative directions, the direction is described without using a positive or negative sign, such as “X direction” or “X axis”. The same applies to the Y direction and the Z direction. In the following description, the direction of gravitational force is shown as a “−Z direction”.

### First Embodiment

### [Outline of Inactivating Apparatus]

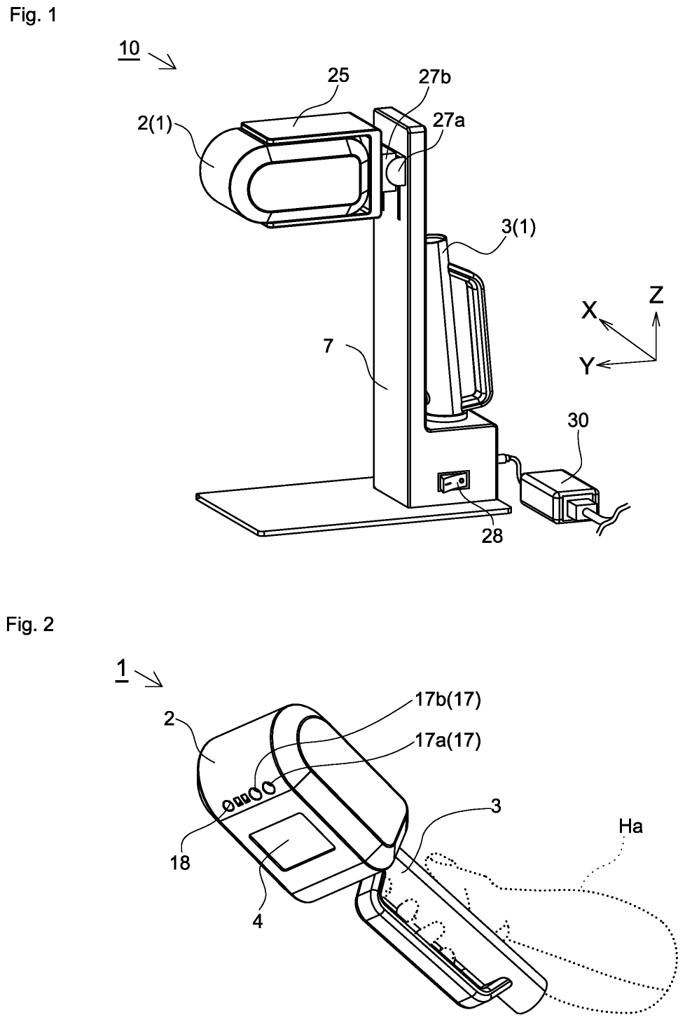

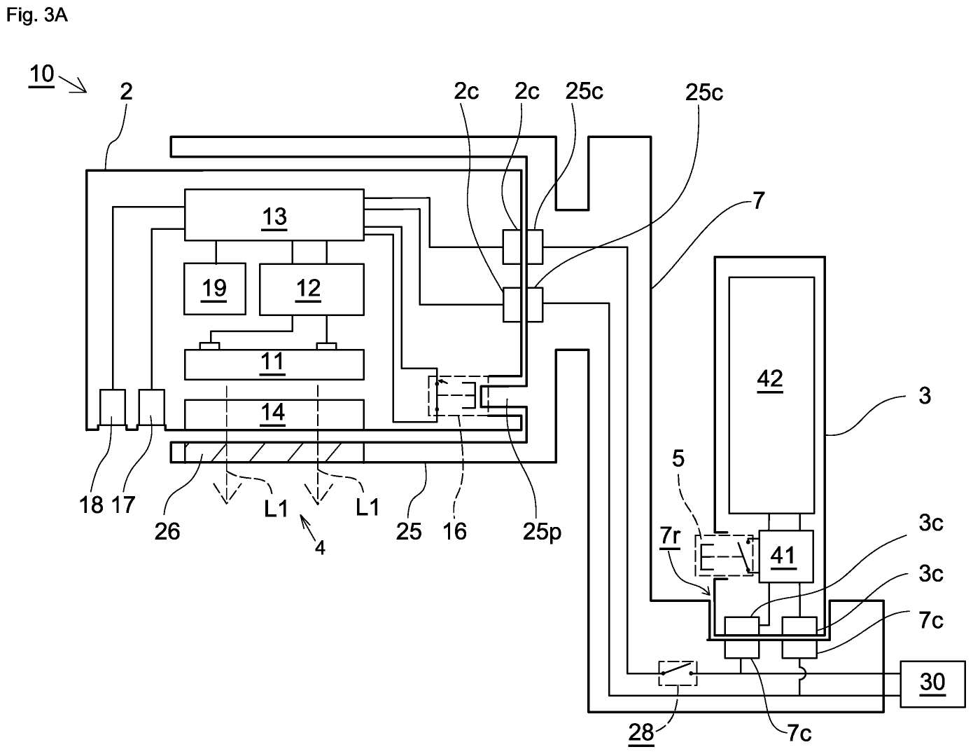

A first embodiment is an inactivating apparatus that can be used by a method in which a light irradiation device of ultraviolet light is used by being fixed on a table and by a method in which the light irradiation device is used by being held in a hand. FIG. 1 is an external view when the light irradiation device is used by being fixed. FIG. 2 is an external view when the light irradiation device is carried and used.

As shown in FIG. 1, an inactivating apparatus 10 includes a light irradiation device 1 that applies ultraviolet light and a fixing base 7 to/from which the light irradiation device 1 is attachable/detachable. The light irradiation device 1 has a main body 2 having a light source (not shown in FIG. 1 and FIG. 2) therein and a handle 3 separable from the main body 2. When the light irradiation device 1 is used by attaching the main body 2 to the fixing base 7, the handle 3 is not used. In this embodiment, the handle 3 is separated from the main body 2 and placed to be fitted in a recess of the fixing base 7.

FIG. 2 shows a case where the light irradiation device 1 is carried by a user. In the inactivating apparatus 10 shown in FIG. 1, the light irradiation device 1 is used by detaching the main body 2 and the handle 3 from the fixing base 7 and attaching the handle 3 to the main body 2. In FIG. 2, a hand Ha of the user holding the light irradiation device 1 is shown by a dotted line. The user holds the handle 3 when using the light irradiation device 1.

The handle 3 is provided with a lighting switch 5 for radiating ultraviolet light. In this embodiment, the light irradiation device 1 emits ultraviolet light from a light extraction part 4 only when the user pushes the lighting switch 5. However, although described later in detail, there is a case where depending on the detection status of a human sensor and a distance sensor, the light irradiation device 1 does not emit ultraviolet light even when the lighting switch 5 is pushed. The light irradiation device 1 may be configured to emit ultraviolet light toward an object to be irradiated for a certain time from when the user pushes the lighting switch 5.

The “certain time” during which ultraviolet light is radiated is a time required to inactivate a target to be inactivated present on the surface of a desired object using the light irradiation device 1 carried by the user. The time required for inactivation varies depending on a separation distance from the light extraction part 4 of the light irradiation device 1 to the surface of the object or the type of target to be inactivated. For example, when the separation distance from the light extraction part 4 of the light irradiation device 1 to the surface of the object is 10 cm, about 3 seconds are required to inactivate viruses present on the surface of the object, and about 30 seconds are required to inactivate bacteria present on the surface of the object.

[Method in which Light Irradiation Device is Used by being Fixed]

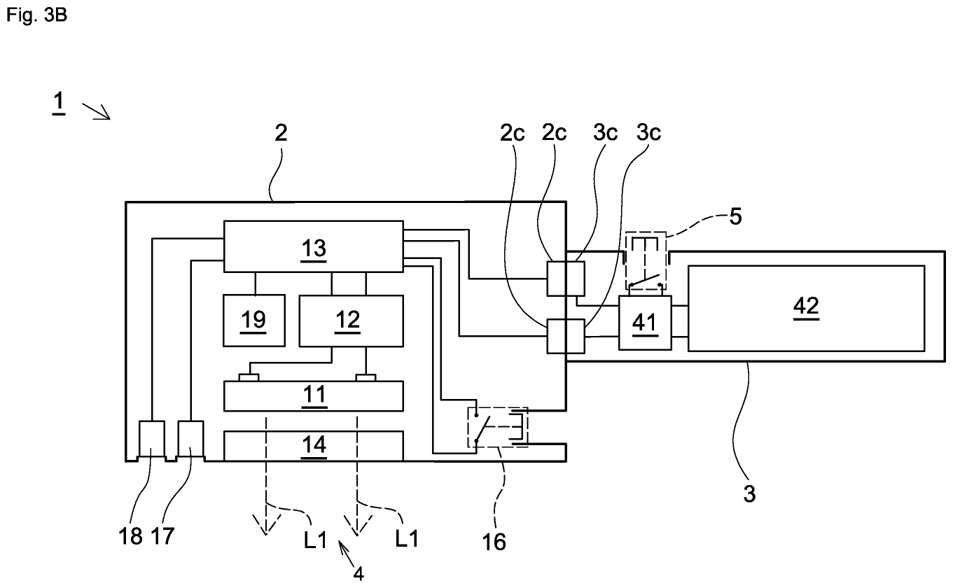

FIG. 3A is a block diagram when the inactivating apparatus 10 according to the first embodiment is used by fixing the main body 2 of the light irradiation device 1. In the block diagram, electrical connections between parts constituting the inactivating apparatus 10 are schematically shown. A line connecting the parts to each other shows a state where the parts are electrically connected.

As shown in FIG. 3A, the light irradiation device 1 has, inside the main body 2, a light source 11, a power source 12 that generates a voltage supplied to the light source 11, a controller 13 that controls a lighting state of the light source 11, an optical filter 14, a distance sensor 17, a human sensor 18, and a cooler 19.

The light irradiation device 1 has a storage battery 42 inside the handle 3. When carried and used, the light irradiation device 1 is operated using the electrical power of the storage battery 42. The storage battery 42 can supply an electrical power of, for example, DC 12V or 24V. The light irradiation device 1 further has a storage battery controller 41 inside the handle 3. The storage battery controller 41 is electrically connected to the lighting switch 5, electrical contact points 3c, and the storage battery 42. The storage battery controller 41 controls discharge and charge of the storage battery 42. It should be noted that even when the light irradiation device 1 is carried and used, the light irradiation device 1 may be connected to an external power source via a cord to be operated by the electrical power of the external power source.

The light source 11 radiates ultraviolet light L1 shown by a dashed arrow. As the light source 11, an excimer lamp, an LED, or an LD is used. An example of the excimer lamp is a KrCl excimer lamp having a light emitting tube filled with a light emitting gas containing KrCl. The KrCl excimer lamp emits ultraviolet light having a main peak wavelength of about 222 nm. As described above, ultraviolet light having an emission wavelength of 190 nm or more and less than 240 nm is safe for human bodies.

Instead of the KrCl excimer lamp, a KrBr excimer lamp may be used which has a light emitting tube filled with a light emitting gas containing KrBr. The KrBr excimer lamp emits ultraviolet light having a main peak wavelength of about 207 nm and is therefore safe for human bodies.

The optical filter 14 functions as a band pass filter that prevents the passage of light in a harmful wavelength band (240 nm or more and less than 280 nm) contained in light radiated from the light source 11. The light source 11 does not always radiate only light having a wavelength safe for human bodies. For example, the KrCl excimer lamp radiates light in the harmful wavelength band although the intensity of the light is very weak. Therefore, the optical filter 14 is disposed over the entire range of the light extraction part 4 from which the light of the light source 11 is extracted. This reduces the irradiation dose of light in the harmful wavelength band.

The optical filter 14 has a dielectric multilayer film obtained by laminating a plurality of layers different in refractive index. The position and shape of the optical filter 14 are not particularly limited. As in the case of this embodiment, the optical filter 14 may be disposed to be separated from the light source 11. Alternatively, the optical filter 14 may be formed integrally with the light source 11.

The fixing base 7 has a main body supporter 25 for supporting the main body 2 (see FIG. 1 and FIG. 3A), an optical member 26 that changes the properties of light emitted from the light irradiation device 1 (see FIG. 3A), and a main power switch 28 (see FIG. 1 and FIG. 3A).

The fixing base 7 supports the main body supporter 25 via angle adjusters (27a, 27b) (see FIG. 1). The angle adjuster 27a allows the main body supporter 25 to rotate around the X axis. This makes it possible to change the inclination of the main body 2 around the X axis. The angle adjuster 27b allows the main body supporter 25 to rotate around the Y axis. This makes it possible to change the inclination of the main body 2 around the Y axis. Further, the angle adjuster 27a can be vertically moved along the fixing base 7, and therefore the height of the main body 2 from the installation surface of the inactivating apparatus 10 can be changed.

The fixing base 7 further has a recess 7r in which the handle 3 is placed by being fitted (see FIG. 3A). When the handle 3 is fitted in the recess 7r, the electrical contact points 3c of the handle 3 come into contact with electrical contact points 7c of the fixing base 7. However, the electrical contact points (3c, 7c) shown in FIG. 3A are slightly separated from each other to make it easy to distinguish the handle 3 and the fixing base 7. When electrical power is supplied from the external power source 30, the storage battery controller 41 charges the storage battery 42.

The main power switch 28 is a switch for supplying/stopping supply of electrical power from the external power source 30 to the main body 2. It should be noted that in this embodiment, the storage battery 42 can be charged even when the main power switch 28 is turned off to stop the supply of electrical power to the main body 2.

The external power source 30 of this embodiment (see FIG. 1 and FIG. 3A) is an AC/DC converter connected to an alternating current commercial power source. However, a specific example of the external power source 30 is not limited thereto. When the light irradiation device 1 is fixed and used, electrical power supplied from the external power source 30 may be used or electrical power supplied from the storage battery 42 may be used.

The main body supporter 25 supports the main body 2 by sandwiching the top and bottom of the main body 2. The main body 2 and the main body supporter 25 are electrically connected via electrical contact points 2c of the main body 2 and electrical contact points 25c of the main body supporter 25. The electrical contact points (2c, 25c) shown in FIG. 3A are slightly separated from each other to make it easy to distinguish the main body 2 and the main body supporter 25.

In order to make it easy to position the main body 2 in the main body supporter 25, the main body supporter 25 and/or the main body 2 may have a guide member (not shown). A specific example of the guide member may be a convex rail guide that allows the main body 2 to be slidably inserted into the main body supporter 25. Further, the main body supporter 25 and/or the main body 2 may have a lock mechanism (not shown) for fixing the main body 2 to the main body supporter 25 to prevent the main body 2 from falling from the main body supporter 25.

The optical member 26 (a hatched area in FIG. 3A) is disposed in the main body supporter 25 to be close to the light extraction part 4. In the inactivating apparatus 10 according to this embodiment, when the main body 2 is attached to the main body supporter 25, ultraviolet light passes through the optical member 26 before emitted from the light extraction part 4. The optical member 26 changes the properties of ultraviolet light, such as light distribution angle or irradiation intensity.

The optical member 26 of this embodiment is a light diffuser lens that functions as a light diffuser. This makes it possible to increase the light distribution angle of ultraviolet light. Although described later in detail, when the light irradiation device 1 is used by being attached to the fixing base 7, there is a case where it is preferred that light is radiated to a wide range unlike a case where the light irradiation device 1 is carried. Therefore, the light distribution angle is increased by the light diffuser so that light can be radiated to a wide range. As the optical member 26, a light diffuser sheet may be used.

The optical member 26 may be a neutral density filter that limits the amount of light passing through it. Although described later in detail, when the light irradiation device 1 is used by being attached to the fixing base 7, there is a case where it is preferred that ultraviolet light having a lower irradiation intensity is radiated as compared to a case where the light irradiation device 1 is carried. Therefore, a neutral density filter may be used to reduce irradiation intensity.

The power source 12 converts electrical power supplied from the controller 13 to a voltage suitable for the light source 11. The parts disposed inside the main body 2, especially the power source 12, generate heat. Therefore, the parts in the main body 2 are cooled by the cooler 19. As the cooler 19, for example, a cooling fan may be used. The parts in the main body 2 are cooled by introducing a low-temperature gas from the outside of the main body 2 and discharging a high-temperature gas in the main body 2 to the outside of the main body 2 through a vent hole (not shown).

The controller 13 is electrically connected to the distance sensor 17, the human sensor 18, the cooler 19, a control switching switch 16, and the external power source 30. The controller 13 switches the method of controlling the lighting state of the light irradiation device 1 between when the light irradiation device 1 is attached to the fixing base 7 and when the light irradiation device 1 is detached from the fixing base 7.

The main body 2 has the control switching switch 16. The control mode of the light irradiation device 1 is switched using the control switching switch 16. In this embodiment, the control switching switch 16 is a push-button type switch. When the push-button type switch is pushed by a protrusion 25p of the main body supporter 25, a circuit including the switch is closed so that the controller 13 recognizes that the light irradiation device 1 has been attached to the fixing base 7 and activates a first control mode. When the push-button type switch is not pushed by the protrusion 25p so that the circuit including the switch is opened, the controller 13 recognizes that the light irradiation device 1 has been separated from the fixing base 7 and performs an activation determining step of a second control mode. The first control mode and the second control mode will be described later in detail.

The distance sensor 17 of this embodiment is of an ultrasonic type. The distance sensor 17 has a transmitter 17a and a receiver 17b (see FIG. 2). Ultrasonic waves are emitted from the transmitter 17a toward an object and their reflected waves are received by the receiver 17b so that a distance D from the main body 2 of the light irradiation device 1 to the object is measured. The distance sensor 17 is not limited thereto and may be, for example, a sensor using electromagnetic waves such as laser light. The intended use of the distance sensor 17 will be described later.

The human sensor 18 is operated by detecting infrared light emitted from a human or an animal. The human sensor 18 will be described later in detail.

[Method in which Light Irradiation Device is Carried and Used]

FIG. 3B is a block diagram of the light irradiation device 1 of a portable type carried and used. In this block diagram, electrical connections between parts constituting the light irradiation device 1 are schematically shown. A line connecting the parts to each other shows a state where the parts are electrically connected. The same contents as described in the section

[Method in which Light Irradiation Device is Used by being Fixed] Will not Repeatedly be Described.

The light irradiation device 1 is obtained by attaching the handle 3 to the main body 2. When the handle 3 is attached to the main body 2, the electrical contact points 2c of the main body 2 come into contact with the electrical contact points 3c of the handle 3. The light irradiation device 1 of this embodiment is operated by the electrical power of the storage battery 42 in the handle 3.

### [Control Method]

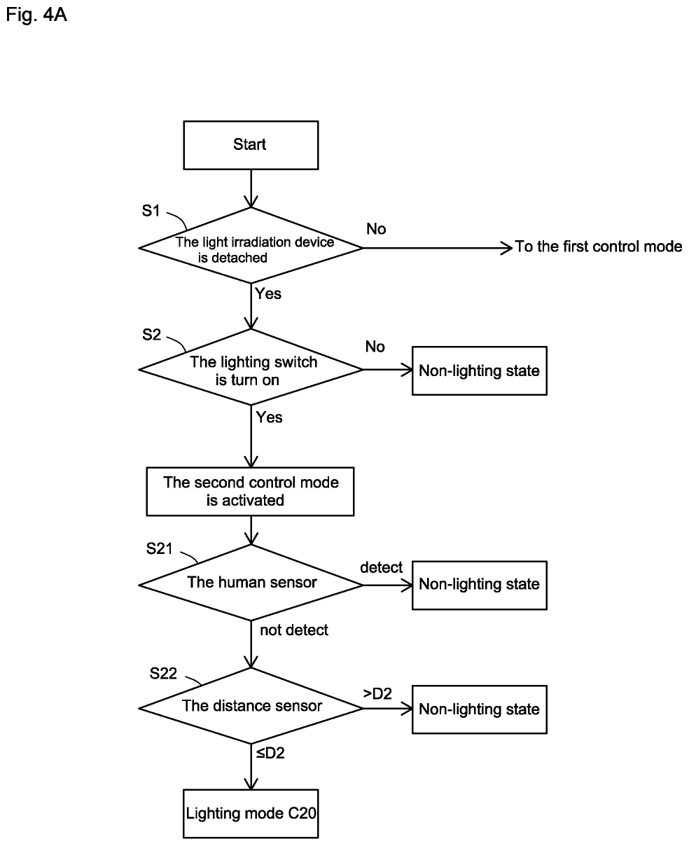

FIG. 4A and FIG. 4B are each a flow diagram showing the control of the inactivating apparatus 10 according to the first embodiment. Referring to FIG. 4A, in a step S1, whether the main body 2 of the light irradiation device 1 is attached to the fixing base 7 or detached from the fixing base 7 is determined by detecting ON/OFF of the control switching switch 16. In this embodiment, when the control switching switch 16 is OFF (when the circuit is opened), the controller 13 recognizes that the main body 2 of the light irradiation device 1 is detached from the fixing base 7. When the control switching switch 16 is ON (when the circuit is closed), the controller 13 recognizes that the main body 2 of the light irradiation device 1 is attached to the fixing base 7.

A switch for switching the control mode is not limited to the control switching switch 16. As a variation, the main body 2 may have another push-button type switch (not shown) for closing the circuit when the handle 3 is attached. When the another push-button type switch is pushed so that the circuit is closed, the controller 13 may recognize that the handle 3 has been attached to the main body 2 and perform the activation determining step (which will be described later in detail) of the second control mode. Alternatively, attachment/detachment of the main body 2 of the light irradiation device 1 to/from the fixing base 7 may be detected by a method other than such a switch.

When recognizing that the main body 2 of the light irradiation device 1 is not detached from the fixing base 7 (in the case of No in FIG. 4A), the controller 13 activates the first control mode. When recognizing that the light irradiation device 1 is detached from the fixing base 7 (in the case of Yes in FIG. 4A), the controller 13 performs a step S2.

The step S2 is a preparation step of the second control mode. In the step S2, the light source 11 is in a non-lighting state when the lighting switch 5 is not pushed (when electrical power is not applied). The second control mode is activated only when the lighting switch 5 is pushed (only when electrical power is applied). As a variation, the second control mode may be activated for a predetermined time (e.g., 10 seconds) after the lighting switch 5 is pushed.

The switching of the control mode has been described above with reference to a case where the control switching switch is used. However, the control mode may be switched without using such a switch, that is, the second control mode may be started when the lighting switch 5 is pushed and the first control mode may be started when the lighting switch 5 is not pushed.

### [First Control Mode]

The first control mode will be described. When attached to the fixing base 7, the light irradiation device 1 is intended to radiate ultraviolet light not only to the surface of an object but also to an environment including a human. The ultraviolet light can inactivate not only pathogens present on the surface of the object but also pathogens suspended in a space.

When ultraviolet light is radiated to an environment including a human, there is a case where the dose of ultraviolet irradiation is required to be reduced. For example, ACGIH (American Conference of Governmental Industrial Hygienists) or JIS Z 8812 (Measuring methods of eye-hazardous ultraviolet radiation) specifies that the dose of ultraviolet irradiation to a human body per day (8 hours) should be equal to or less than a threshold limit value (TLV) dependent on wavelength.

As described above, the wavelength of emitted light used in the present invention is very low hazardous to human bodies. However, in order to further increase safety, it is desirable that the dose of ultraviolet irradiation is set to satisfy the above-described specified TLV. Therefore, in this embodiment, when ultraviolet light is radiated in the first control mode, a specified value is set on the basis of the TLV as a requirement for ultraviolet irradiation to an environment including a human, and the irradiation intensity and cumulative irradiation time of ultraviolet light are controlled so that the dose of ultraviolet irradiation does not exceed the specified value.

Therefore, in the first control mode, irradiation intensity is lower as compared to the second control mode described later, and the light irradiation device 1 is turned on for a long time. The “long time” herein refers to cumulative time during turn-on and includes not only a continuous lighting state but also a state in which turn-on and turn-off are intermittently repeated in a short time. In the first control mode, ultraviolet light is radiated to a wider range as compared to the second control mode.

An example of the procedure of the first control mode will be described with reference to FIG. 4B. In a step S11, the distance sensor 17 measures a distance D from the main body 2 to an object to be irradiated. In order to prevent only a narrow area in the object to be irradiated from being continuously irradiated with ultraviolet light, the light irradiation device 1 is turned off when the distance sensor 17 detects the object nearer than a first specified value D1 (i.e., when the distance D from the main body 2 to the closest object is smaller than a first specified value D1). When the distance sensor 17 does not detect the object nearer than the first specified value D1 (i.e., when the distance D from the main body 2 to the closest object is equal to or larger than the first specified value D1), a step S12 is performed. The first specified value D1 should be 5 cm or more and 200 cm or less. However, when the optical member 26 prevents only a narrow area in the object to be irradiated from being irradiated with ultraviolet light or when the distance from the main body 2 to the object to be irradiated is nearly fixed, distance determination in the step S11 may be omitted.

The step S12 is a setting selecting step. In the step S12, the setting of the light irradiation device 1 is selected on the basis of setting conditions (here, a first setting C1 to a third setting C3) previously set by the installation personnel of the inactivating apparatus 10. A step S13 is a human detecting step. The operation of the light irradiation device 1 is different between when the human sensor detects a human and when the human sensor does not detect a human. The details of the step S13 will be described according to the setting condition of the step S12.

### (First Setting C1)

When the light irradiation device 1 is disposed in an unattended environment, the installation personnel previously performs setting so that the controller 13 selects the first setting C1. For example, when the inactivating apparatus 10 is of a desktop type or a floor-standing type as in the case of this embodiment, the light irradiation device 1 may be positioned near a human. In this case, when the human sensor 18 detects a human, the light irradiation device 1 is turned off to further increase safety. When a human is away from the light irradiation device 1 and the human sensor 18 does not detect the human, the light irradiation device 1 is turned on in a lighting mode C10. In the lighting mode C10, ultraviolet light is not radiated to a human and therefore may be radiated at an irradiation intensity such that the dose of ultraviolet irradiation exceeds the specified value based on TLV.

### (Second Setting C2)

When the light irradiation device 1 is fixed at a position slightly away from a human, the installation personnel previously performs setting so that the controller 13 selects the second setting C2. For example, when the inactivating apparatus 10 is disposed 1 m or less above the top of a human, the light irradiation device 1 is positioned slightly away from the human. In the case of this position, when the human sensor 18 detects a human, the light irradiation device 1 is turned on in a lighting mode C12. In the lighting mode C12, irradiation intensity is weaker or irradiation time is shorter as compared to a lighting mode C11. When a human is away from the light irradiation device 1 and the human sensor 18 does not detect the human, the light irradiation device 1 is turned on in the lighting mode C11. In the lighting mode C11, irradiation intensity is weaker or irradiation time is shorter as compared to C10.

### (Third Setting C3)

When the light irradiation device 1 is fixed at a position far away from a human, the installation personnel previously perform setting so that the controller 13 selects the third setting C3. For example, when the inactivating apparatus 10 is disposed more than 1 m above the top of a human (e.g., at a high position on a wall or column or on a high ceiling), the light irradiation device 1 is positioned far away from the human. In this case, when the human sensor 18 detects a human, the light irradiation device 1 is turned on in a lighting mode C13. In the lighting mode C13, irradiation intensity is weaker as compared to the lighting mode C11 but stronger as compared to the lighting mode C12 or irradiation time is shorter as compared to the lighting mode C11 but longer as compared to the lighting mode C12. When a human is away from the light irradiation device 1 and the human sensor 18 does not detect the human, the light irradiation device 1 is turned on in the lighting mode C11.

Ultraviolet light emitted from the light irradiation device 1 has an emission wavelength of 190 nm or more and less than 240 nm, and is therefore safe for human bodies and contains little light in a harmful wavelength band (240 nm or more and less than 280 nm). However, in order to pursue more safety for a user, as described above, the irradiation intensity of the light source 11 may be set to increase as the distance from the position where the light irradiation device 1 is fixed to the position where a human may be present increases.

In the above description, three levels are set as the first setting C1 to the third setting C3, but four or more levels may be set as setting conditions. Further, the setting selection has been described above with reference to a case where the controller 13 selects a setting according to the setting condition previously set by the installation personnel of the inactivating apparatus 10, but is not limited thereto. For example, the controller 13 may select any one of the first setting C1 to the third setting C3 according to the measurement result of the distance sensor 17 (the distance D from the light irradiation device 1 to the object to be irradiated) obtained in the step S11.

### [Second Control Mode]

The second control mode will be described. When the light irradiation device 1 can be carried by a user, in order to inactivate a target to be inactivated present on the surface of a desired object, the user brings the light irradiation device 1 close to the surface to irradiate the surface with ultraviolet light. In the second control mode, irradiation intensity is higher as compared to the first control mode, and the light irradiation device 1 is turned on for only a limited short time (e.g., 3 to 30 seconds). Since the light irradiation device 1 is turned on for a short time and ultraviolet light is not radiated to a human, ultraviolet light may be radiated at an irradiation intensity such that the dose of ultraviolet irradiation exceeds the specified value based on TLV. Further, since only the limited object surface is irradiated with ultraviolet light, the light distribution angle of ultraviolet light in the second control mode should be smaller than that in the first control mode. The light distribution angle in the second control mode is, for example, 70 degrees or less.

Referring to FIG. 4A again, an example of the procedure of the second control mode will be described. In a step S21, when the human sensor 18 detects a human, the light irradiation device 1 is put into a non-lighting state. When the human sensor 18 does not detect a human, a step S22 is performed.

In the second control mode, the light irradiation device 1 is carried and used, and therefore a human is present near the light irradiation device 1. However, the human sensor 18 is disposed in a recess of the hosing of the main body 2, and the detection range of the human sensor 18 is oriented close to a range irradiated with ultraviolet light. Therefore, when the light irradiation device 1 is used in such a manner that the range irradiated with ultraviolet light is not directed toward a human, the human sensor 18 does not detect a human.

In the step S22, a distance D from the main body 2 to an object to be irradiated is measured. When carried and used, the light irradiation device 1 is brought close to the object to be irradiated to inactivate a specific target object. When the distance sensor 17 does not detect the object nearer than a second specified value D2 (i.e., when the distance D from the main body 2 to the closest object is larger than a second specified value D2), the light irradiation device 1 should be turned off to prevent ultraviolet light from leaking to a range wider than the specific target object.

When the distance sensor 17 detects the object nearer than the second specified value D2 (i.e., when the distance D from the main body 2 to the closest object is equal to or less than the second specified value D2), the light irradiation device 1 should be turned on in a lighting mode C20. The second specified value D2 is preferably 5 cm or more and 100 cm or less, more preferably 5 cm or more and 50 cm or less, even more preferably 10 cm or more and 30 cm or less. The second specified value D2 may be the same as or different from the first specified value D1.

Irradiation with ultraviolet light in the second control mode is short-time irradiation of an object other than a human, and therefore irradiation intensity in the lighting mode C20 may be set so that the dose of ultraviolet irradiation exceeds a specified value set on the basis of a requirement for ultraviolet irradiation to an environment including a human.

As described above, in the second control mode, the light irradiation device 1 is put into a lighting state when the distance D detected by the distance sensor 17 is equal to or less than a predetermined distance, and in the first control mode, the light irradiation device 1 is put into a non-lighting state when the distance D detected by the distance sensor 17 is equal to or less than a predetermined distance. The first control mode and the second control mode are opposite in the lighting control of the light irradiation device 1 when the distance sensor 17 detects/does not detect an object.

The flow diagrams shown in FIG. 4A and FIG. 4B are schematic diagrams not showing a procedure after the selection of turn-on or turn-off. Actually, the light irradiation device 1 is repeatedly controlled by returning to the step S1 even after the selection of turn-on or turn-off. For example, when the light irradiation device 1 is detached from the fixing base 7 while turned on by being attached to the fixing base 7, the first control mode is switched to the activation determining step of the second control mode. For example, when the human sensor 18 detects a human while the light irradiation device 1 is turned on in the second control mode, the light irradiation device 1 is put into a non-lighting state.

### Second Embodiment

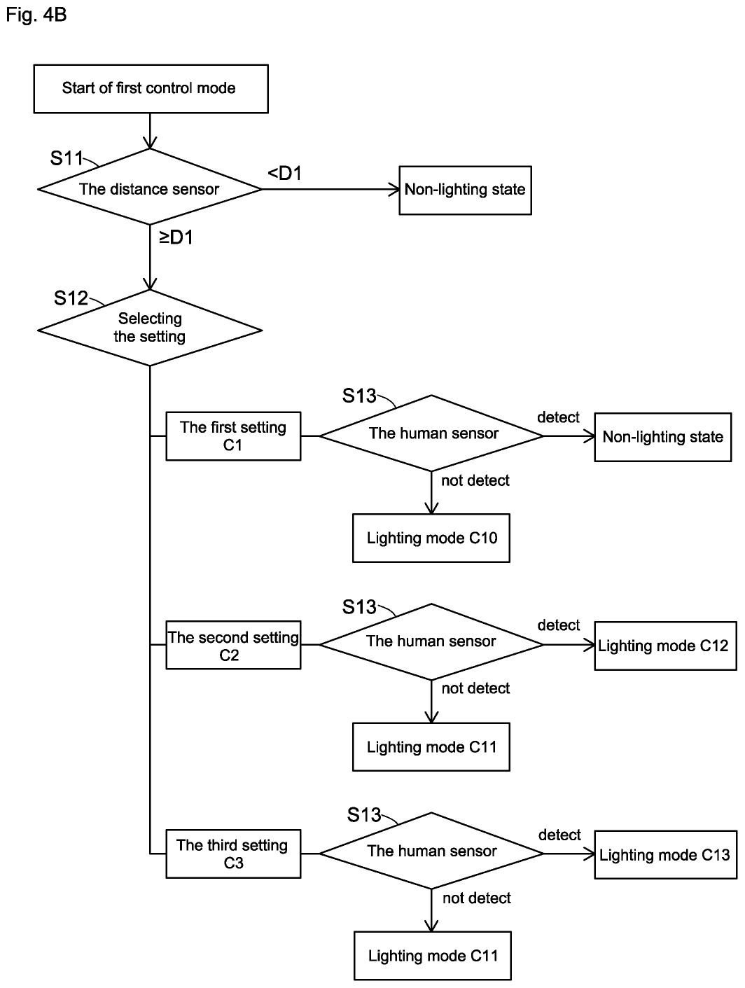

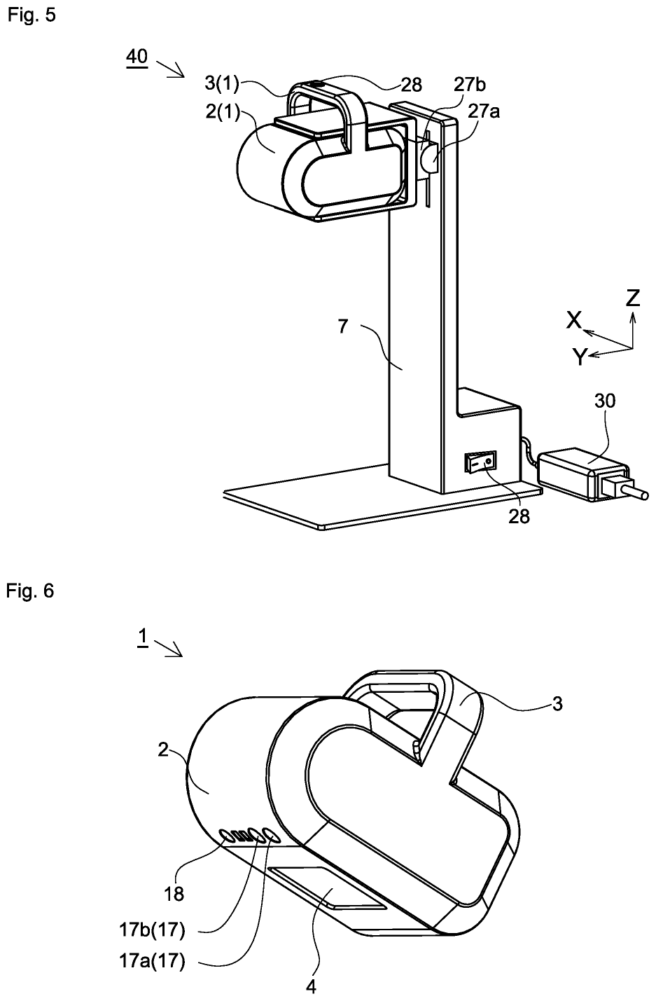

An inactivating apparatus according to a second embodiment will be described with reference to FIG. 5 and FIG. 6. The second embodiment will be described below by focusing differences from the first embodiment. The second embodiment other than the points described below has the same structure as the first embodiment described above and can be implemented in the same manner. The same applies to the third and later embodiments.

As shown in FIG. 5 and FIG. 6, the light irradiation device 1 of an inactivating apparatus 40 according to the second embodiment is not configured in such a manner that the main body 2 can be separated from the handle 3. Therefore, the light irradiation device 1 can be attached to the fixing base 7 without separating the handle 3 from the main body 2. In this embodiment, the storage battery 42 and the storage battery controller 41 are housed in the main body 2. The storage battery controller 41 may be integrally formed with the controller 13.

The lighting switch 5 is provided in the handle 3 at a position where a user can operate it with a hand holding the light irradiation device 1. Alternatively, the lighting switch 5 may be disposed at a position where the lighting switch 5 cannot be pushed when the light irradiation device 1 is attached to the fixing base 7. When the lighting switch 5 is disposed at such a position, it is possible to prevent the light irradiation device 1 from being turned on at a high irradiation intensity used in the second control mode when the control mode is not switched from the second control mode to the first control mode due to some problems in spite of the fact that the light irradiation device 1 is attached to the fixing base 7.

### Third Embodiment

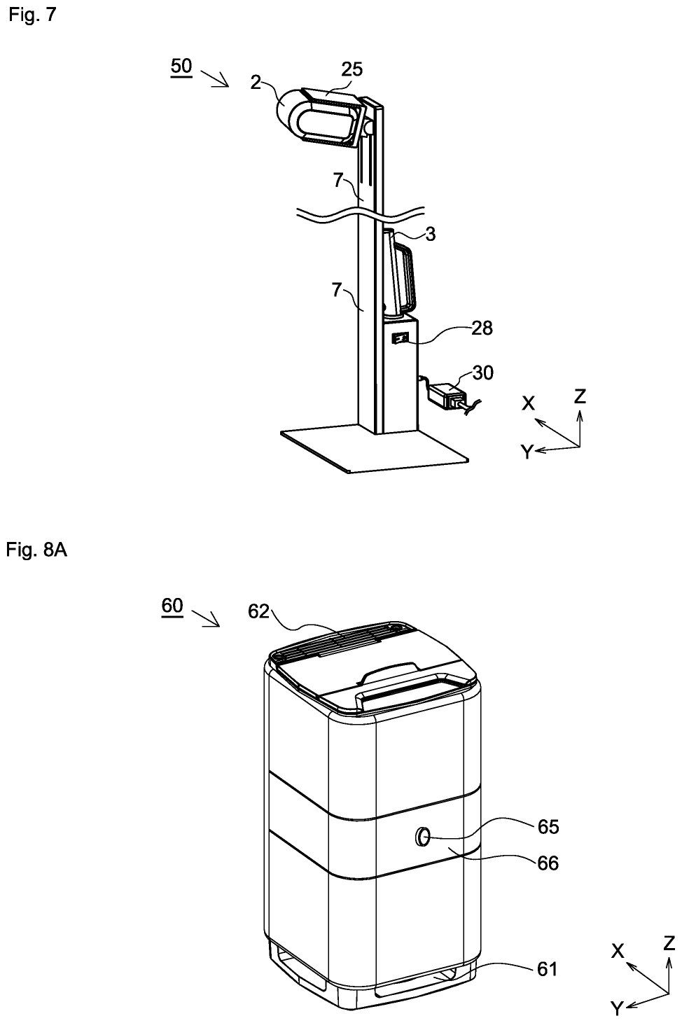

An inactivating apparatus 50 according to a third embodiment will be described with reference to FIG. 7. The inactivating apparatus 50 is placed not on a table but on a floor. The fixing base 7 is a pole longer than the body height of a human. The main body 2 of the light irradiation device 1 emits ultraviolet light from a position higher than the body height of a human. As a variation of the inactivating apparatus 50, the fixing base 7 may be attached to a column or wall. Alternatively, the fixing base 7 may be attached to a ceiling. The fixing base 7 may be integrally formed with a table, a floor, a wall, a column, or a ceiling to be part of the table, the floor, the wall, the column, or the ceiling. The inactivating apparatus 50 may be placed not indoors but outdoors.

### Fourth Embodiment

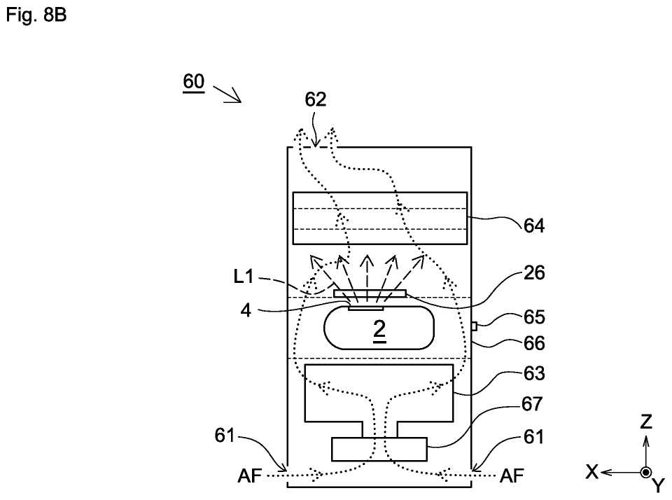

An inactivating apparatus according to a fourth embodiment will be described with reference to FIG. 8A and FIG. 8B. The inactivating apparatus is fixed to equipment. In this embodiment, the equipment is an air purifier. FIG. 8A is an external view of an example of an air purifier including the inactivating apparatus, and FIG. 8B is a conceptual diagram of the air purifier.

An air purifier 60 according to this embodiment has an air inlet 61 at the bottom and an air outlet 62 at the top. The flow of air in the air purifier 60 is shown by a dashed arrow marked with AF. The flow of air AF passes through a coarse filter 67, a blower 63, and a fine filter 64 and exits from the air outlet 62. The main body 2 of an ultraviolet light irradiation device is disposed between the blower 63 and the fine filter 64 so that the light extraction part 4 faces upward, and ultraviolet light L1 is radiated to the flow of air AF moving toward the fine filter 64. The ultraviolet light L1 is diffused by the optical member 26. In this way, pathogens present in the flow of air AF in the air purifier 60 are inactivated.

In this embodiment, the main body 2 of the light irradiation device 1 is fixed in an internal space of the air purifier 60, that is, in an environment isolated from a space where a human is present. Therefore, the dose of ultraviolet irradiation does not have to be set to satisfy the above-described specified TLV. Further, the light source 11 used in the inactivating apparatus according to this embodiment does not have to emit ultraviolet light having an emission wavelength of 190 nm or more and less than 240 nm.

When a handle 65 of the air purifier 60 is pulled, a center part 66 of housing of the air purifier 60 can be drawn out. Although not shown in FIG. 8B, the main body 2 is detachably attached to the fixing base inside the air purifier 60. The fixing base is fixed to the center part 66 of the housing. The main body 2 can be taken out of the air purifier 60 by drawing out the center part 66 of the housing. The main body 2 taken out of the air purifier 60 can be used as a portable inactivating apparatus by attaching a handle (not shown) thereto.

The light irradiation device 1 can be incorporated in equipment other than the air purifier 60. For example, the light irradiation device 1 may be incorporated in a cooling and heating machine or a vacuum cleaner.

The inactivating apparatus according to each of the embodiments has been described above. However, the present invention is not limited to the embodiments described above, and two or more of the embodiments and the variations may be combined and various changes or modifications may be made to the embodiments without departing from the spirit of the present invention.

What is claimed is:

1. An inactivating apparatus comprising:
a light irradiation device having a light source that emits ultraviolet light for inactivating pathogens;
a fixing base to/from which a main body of the light irradiation device is attachable/detachable; and
a controller that switches control of a lighting state of the light irradiation device between when the main body is attached to the fixing base and when the main body is detached from the fixing base.

2. The inactivating apparatus according to claim 1, wherein
the light irradiation device further has a lighting switch for turning on the light source, and
the controller activates a first control mode irrespective of a state of the lighting switch when the light irradiation device is attached to the fixing base, and activates a second control mode when the light irradiation device is detached from the fixing base and a user of the inactivating apparatus turns on the lighting switch.

3. The inactivating apparatus according to claim 2, wherein the controller controls, in the first control mode, irradiation intensity and irradiation time of the ultraviolet light so that a specified value set on a basis of a requirement for ultraviolet irradiation to an environment including a human is not exceeded, and controls, in the second control mode, irradiation intensity of the light source so that the specified value is exceeded.

4. The inactivating apparatus according to claim 1, wherein the light irradiation device has a detector capable of detecting detaching of the light irradiation device from the fixing base.

5. The inactivating apparatus according to claim 1, wherein
the light irradiation device further has a human sensor, and
when the human sensor detects a human in a second control mode, the light source is not turned on.

6. The inactivating apparatus according to claim 1, wherein
the light irradiation device further has a distance sensor, and
the controller turns on the light source when the distance sensor does not detect an object nearer than a first specified value in a first control mode, and turns on the light source when the distance sensor detects an object nearer than a second specified value in a second control mode.

7. The inactivating apparatus according to claim 1, wherein a light distribution angle of emitted light from the light irradiation device when the light irradiation device is attached to the fixing base is larger than that when the light irradiation device is detached from the fixing base.

8. The inactivating apparatus according to claim 7, wherein
the fixing base has a light diffuser, and
when the light irradiation device is attached to the fixing base, the light distribution angle is increased by passage of the emitted light from the light irradiation device through the light diffuser.

9. The inactivating apparatus according to claim 1, wherein irradiation intensity of the ultraviolet light when the light irradiation device is attached to the fixing base is lower than that when the light irradiation device is detached from the fixing base.

10. The inactivating apparatus according to claim 1, wherein
the fixing base has a neutral density filter, and
when the light irradiation device is attached to the fixing base, irradiation intensity of emitted light from the light irradiation device is reduced by passage through the neutral density filter.

11. The inactivating apparatus according to claim 1, wherein
the light irradiation device further has a handle separable from the main body of the light irradiation device, and
the handle has a storage battery for turning on the light source.

12. The inactivating apparatus according to claim 1, wherein the fixing base is disposed on a table, a floor, a wall, a column, or a ceiling.

13. The inactivating apparatus according to claim 1, wherein the fixing base is part of a table, a floor, a wall, a column, or a ceiling.

14. The inactivating apparatus according to claim 1, wherein when the light irradiation device is attached to the fixing base, emitted light from the light irradiation device is radiated to an environment isolated from a space where a human is present.

15. A light irradiation device having a light source that emits ultraviolet light for inactivating pathogens, the light irradiation device comprising:
a light source that emits ultraviolet light for inactivating pathogens; and
a controller that switches control of lighting state of the light irradiation device between when the light irradiation device is attached to the fixing base and when the light irradiation device is detached from the fixing base, and being configured to be attachable/detachable to/from the fixing base.

16. An inactivating apparatus comprising:
a light irradiation device having a light source that emits ultraviolet light for inactivating pathogens; and
a fixing base to/from which the light irradiation device is attachable/detachable and which has at least one of a light diffuser and a neutral density filter,
wherein when the light irradiation device is attached to the fixing base, emitted light from the light irradiation device passes through at least one of the light diffuser and the neutral density filter.
